(19) United States
(12) Patent Application Publication
(10) Pub. No.: US 2022/0142638 A1
(43) Pub. Date: May 12, 2022

(54) DEVICES FOR ADJUSTABLY TENSIONING SUTURES, AND ASSOCIATED SYSTEMS AND METHODS

(71) Applicant: Inari Medical, Inc., Irvine, CA (US)

(72) Inventors: Paige Elizabeth Enright, Costa Mesa, CA (US); Thomas M. Tu, Louisville, KY (US); Jaqueline Macias, Fullerton, CA (US)

(21) Appl. No.: 17/523,565

(22) Filed: Nov. 10, 2021

Related U.S. Application Data
(60) Provisional application No. 63/111,774, filed on Nov. 10, 2020.

(51) Int. Cl. A61B 17/04

(52) U.S. Cl. CPC A61B 17/0466; A61B 2017/0496

(57) ABSTRACT

Disclosed herein are devices, systems, and methods for adjustably tensioning sutures, such as sutures used to close a catheter access site of a human patient. In some embodiments, a suture tensioning device includes a housing and an actuation member slidably coupled to the housing. The housing can include a first through-hole and a second through-hole at least partially aligned along an axis. The actuation member can include a third-through-hole. The suture tensioning device can further include a biasing member operably coupled between the actuation member and the housing. The biasing member can bias the actuation member to a first position in which the third through-hole is offset from the axis. The actuation member is movable to a second position in which the third through-hole is at least partially aligned with the first and second through-holes along the axis.

### CROSS-REFERENCE TO RELATED APPLICATION

This application claims the benefit of U.S. Provisional Patent Application No. 63/111,774, filed Nov. 10, 2020, and titled “DEVICES FOR ADJUSTABLY TENSIONING SUTURES, AND ASSOCIATED SYSTEMS AND METHODS,” which is incorporated herein by reference in its entirety.

### TECHNICAL FIELD

The present technology generally relates to systems, methods, and devices for adjustably tensioning sutures, such as sutures used to close a percutaneous catheter access site of a human patient.

### BACKGROUND

Percutaneous medical procedures involve accessing the inner organs, tissue, and/or vasculature of a patient via a puncture in the skin of the patient. A percutaneous approach is commonly used in intravascular procedures such as angioplasty, stenting, clot removal, and so on. More specifically, many intravascular procedures include inserting a catheter and/or medical device into a blood vessel of a patient through an access site, such as a popliteal access site, an internal jugular access site, a femoral access site, or another venous or arterial access site. For example, an introducer assembly can be positioned in the blood vessel through the access site, and one or more medical instruments can be advanced through the introducer assembly for carrying out the intravascular procedure.

After such procedures, the access site must be closed to maintain hemostasis as the patient recovers. Common techniques for closing the access site include manual compression and suturing. However, it may be difficult to successfully close the access site and achieve hemostasis when the access site is large, such as for intravascular procedures utilizing large bore catheters. Additionally, the access site closure may be uncomfortable to the patient during recovery.

### BRIEF DESCRIPTION OF THE DRAWINGS

Many aspects of the present technology can be better understood with reference to the following drawings. The components in the drawings are not necessarily to scale. Instead, emphasis is placed on illustrating clearly the principles of the present disclosure.

FIGS. 1A and 1B are an isometric view and a partially-exploded isometric view, respectively, of a suture tensioning device in accordance with embodiments of the present technology.

FIGS. 1C and 1D are exploded top and bottom isometric views, respectively, of the tensioning assembly 102 in accordance with embodiments of the present technology.

FIG. 1E is an exploded isometric view of a dressing assembly of the suture tensioning device in accordance with embodiments of the present technology.

FIG. 2 is an isometric view of a threading tool for use with the suture tensioning device in accordance with embodiments of the present technology.

FIG. 3 is a flow diagram of a process or method for operating the suture tensioning device in accordance with embodiments of the present technology.

FIGS. 4A and 4B are isometric views of the suture tensioning device illustrating threading of a suture therethrough in accordance with embodiments of the present technology.

FIG. 5 is an enlarged side cross-sectional view of a suture positioned within the tensioning assembly of the suture tensioning device in accordance with embodiments of the present technology.

FIG. 6 is an exploded isometric view of a tensioning assembly in accordance with additional embodiments of the present technology.

FIGS. 7A and 7B are an exploded isometric view and an isometric view, respectively, of a suture tensioning device in accordance with additional embodiments of the present technology.

FIG. 8 is an exploded isometric view of a tensioning assembly in accordance with additional embodiments of the present technology.

FIG. 9 is an exploded isometric view of a suture tensioning device in accordance with additional embodiments of the present technology.

FIGS. 10A and 10B are isometric views of a tensioning assembly in a first position and a second position, respectively, in accordance with additional embodiments of the present technology.

FIG. 11 is an isometric view of a tensioning assembly in accordance with additional embodiments of the present technology.

FIG. 12 is an exploded isometric view of a tensioning assembly in accordance with additional embodiments of the present technology.

FIGS. 13A and 13B are isometric views of a tensioning assembly in a first position and a second position, respectively, in accordance with additional embodiments of the present technology.

### DETAILED DESCRIPTION

The present technology is generally directed to devices for adjustably tensioning sutures, such as sutures used to close a catheter access site of a human patient, and associated systems and methods. In some embodiments, a suture tensioning device includes a housing and an actuation member slidably coupled to the housing. The housing can include a first through-hole and a second through-hole at least partially aligned with one another along an axis. The actuation member can include a third-through-hole. The suture tensioning device can further include a biasing member operably coupled between the actuation member and the housing. The biasing member can exert a biasing force against the actuation member that holds the actuation member in a first position in which the third through-hole is offset from the axis and therefore not aligned with the first and second through-holes of the housing. The actuation member can be movable against the biasing force of the biasing member to a second position in which the third through-hole is at least partially aligned with the first and second through-holes along the axis.

In operation, a user can thread a suture through the first, second, and third through-holes when the actuation member is in the second position. The suture can be used to close a puncture in the patient, such as a catheter access site. After adjusting the tension in the suture to a desired tension, the user can release the actuation member to permit the biasing member to drive the actuation member to the first position. In the first position, the actuation member and/or the housing engage the suture to inhibit the suture from moving through the first, second, and third through-holes. Accordingly, the suture tensioning device can maintain the desired tension in the suture. To adjust the tension in the suture, the user can actuate (e.g., depress) the actuation member against the biasing force of the biasing member to move the actuation member to the second position. The user can then adjust the tension in the suture to another desired tension, and then release the actuation member to lock the suture in position at the selected tension. Accordingly, in one aspect of the present technology the suture tensioning device allows the tension in the suture to be easily adjusted. For example, the user can increase the tension to better close the puncture and maintain hemostasis and/or decrease the tension to accommodate patient comfort.

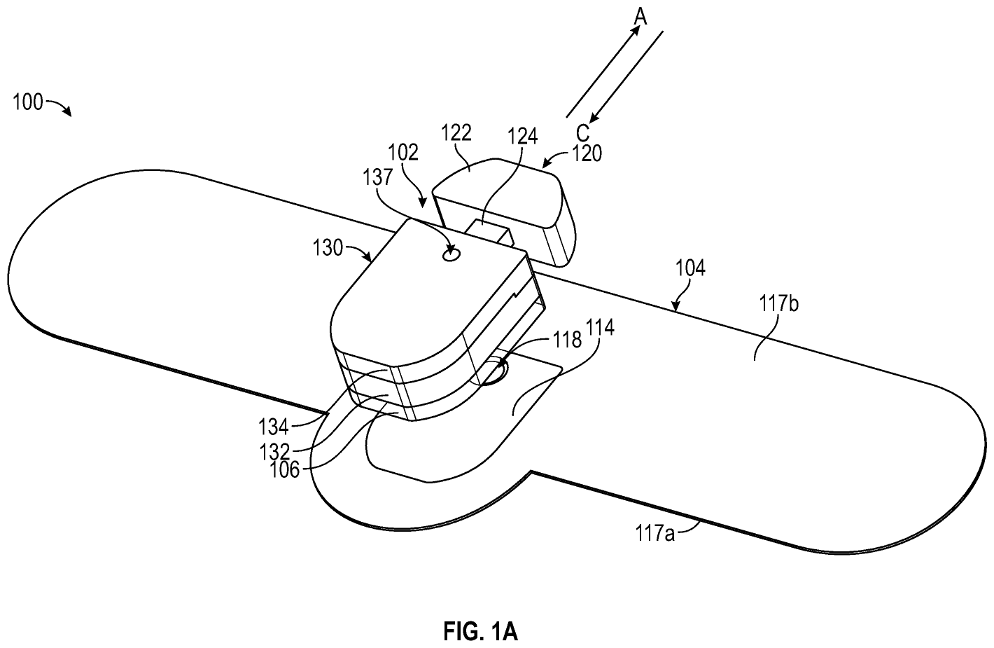

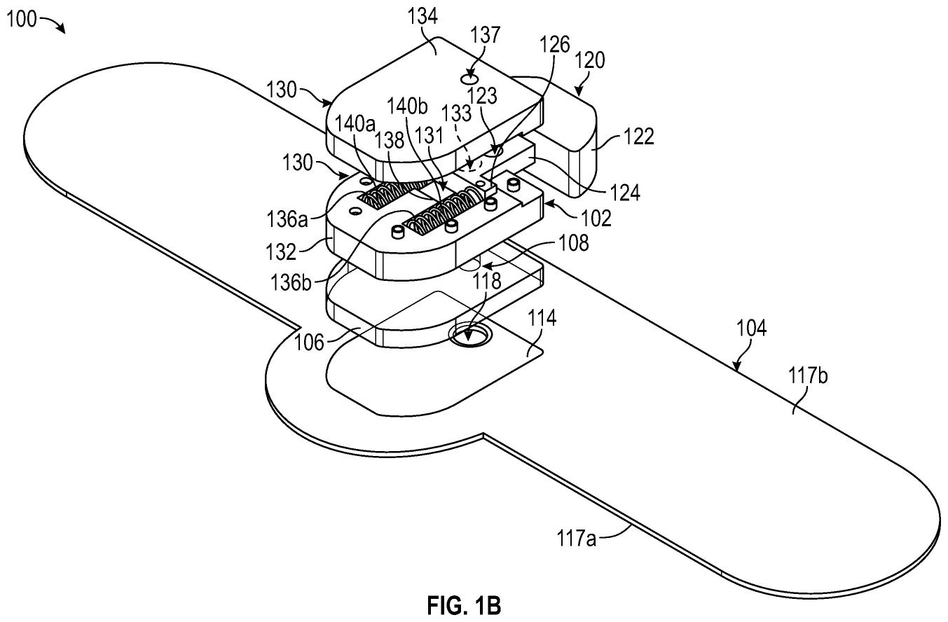

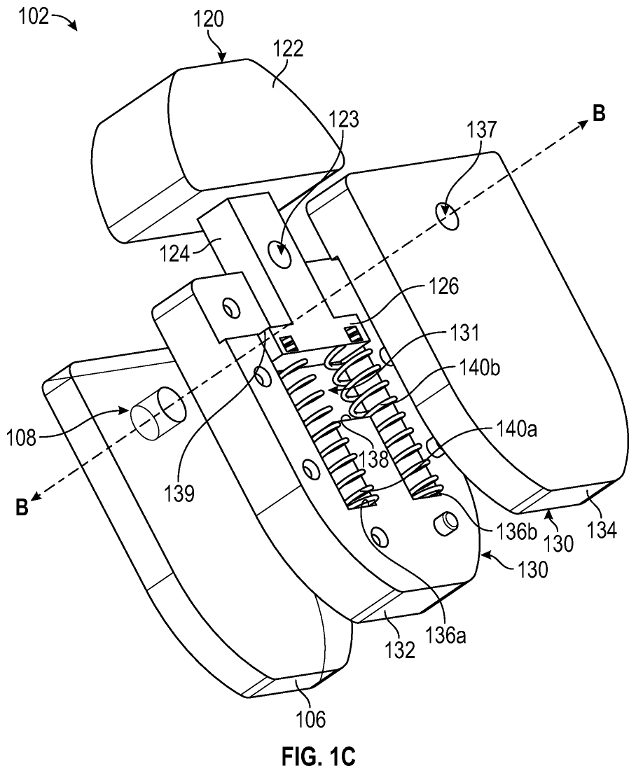

Certain details are set forth in the following description and in FIGS. 1-13B to provide a thorough understanding of various embodiments of the present technology. In other instances, well-known structures, materials, operations, and/or systems often associated with intravascular procedures, percutaneous access sites, etc., are not shown or described in detail in the following disclosure to avoid unnecessarily obscuring the description of the various embodiments of the technology. Those of ordinary skill in the art will recognize, however, that the present technology can be practiced without one or more of the details set forth herein, and/or with other structures, methods, components, and so forth.

The terminology used below is to be interpreted in its broadest reasonable manner, even though it is being used in conjunction with a detailed description of certain examples of embodiments of the technology. Indeed, certain terms may even be emphasized below; however, any terminology intended to be interpreted in any restricted manner will be overtly and specifically defined as such in this Detailed Description section.

The accompanying Figures depict embodiments of the present technology and are not intended to be limiting of its scope unless expressly indicated. The sizes of various depicted elements are not necessarily drawn to scale, and these various elements may be enlarged to improve legibility. Component details may be abstracted in the Figures to exclude details such as position of components and certain precise connections between such components when such details are unnecessary for a complete understanding of how to make and use the present technology. Many of the details, dimensions, angles and other features shown in the Figures are merely illustrative of particular embodiments of the disclosure. Accordingly, other embodiments can have other details, dimensions, angles and features without departing from the present technology. In addition, those of ordinary skill in the art will appreciate that further embodiments of the present technology can be practiced without several of the details described below.

With regard to the terms “distal” and “proximal” within this description, unless otherwise specified, the terms can reference a relative position of the portions of a catheter subsystem with reference to an operator and/or a location in the vasculature. Also, as used herein, the designations “rearward,” “forward,” “upward,” “downward,” etc., are not meant to limit the referenced component to a specific orientation. It will be appreciated that such designations refer to the orientation of the referenced component as illustrated in the Figures; the systems of the present technology can be used in any orientation suitable to the user.

FIGS. 1A and 1B are an isometric view and a partially-exploded isometric view, respectively, of a suture tensioning device 100 in accordance with embodiments of the present technology. Referring to FIGS. 1A and 1B together, the suture tensioning device 100 can include a tensioning assembly 102 (shown exploded in FIG. 1B) configured to be coupled to a dressing assembly 104 via a pad 106. The dressing assembly 104 is configured to be positioned over an incision or puncture in a patient (e.g., a human patient), such as a percutaneous access site, and can be adhered to the patient (e.g., the skin of the patient) around the puncture. The tensioning assembly 102 is configured to receive and tension one or more sutures used to close the puncture. FIGS. 1C and 1D are exploded top and bottom isometric views, respectively, of the tensioning assembly 102 in accordance with embodiments of the present technology. The pad 106 is shown as partially transparent in FIGS. 1A-1D for clarity. FIG. 1E is an exploded isometric view of the dressing assembly 104 in accordance with embodiments of the present technology.

Referring first to FIG. 1E, in some embodiments the dressing assembly 104 includes a contact layer 110 and an adhesive portion 112 over and/or integrated with the contact layer 110. The contact layer 110 and the adhesive portion 112 are shown as partially transparent in FIG. 1E for the sake of clarity. In the illustrated embodiment, the dressing assembly 104 has a generally elongate, oval-like shape while, in other embodiments, the dressing assembly 104 can have other shapes (e.g., rectangular, circular, rectilinear, polygonal, irregular). In some embodiments, the contact layer 110 includes and/or comprises an adhesive material (e.g., an acrylic and/or acrylate material) on its underside for releasably securing the dressing assembly 104 to a patient. In some embodiments, the contact layer 110 (e.g., an upper surface of the contact layer 110) can include a medical tape including, for example, a polyethylene material. The adhesive portion 112 can comprise an acrylic, acrylate, or other suitable adhesive material. In the illustrated embodiment, the dressing assembly 104 includes a first removable protection layer 114a and a second removable protection layer 114b (collectively “protection layer 114”) coupled to the contact layer 110 (e.g., a lower surface of the contact layer 110). The protection layer 114 can cover, seal, and/or protect the contact layer 110 and its adhesive before the dressing assembly 104 is used, and can be removed before placing the dressing assembly 104 on the patient. In some embodiments, the first removable protection layer 114a and the second removable protection layer 114b can be folded to facilitate their removal from the contact layer 110. In other embodiments, the dressing assembly 104 can include more or fewer layers and/or can include different materials. For example, the dressing assembly 104 can include multiple different layers laminated together.

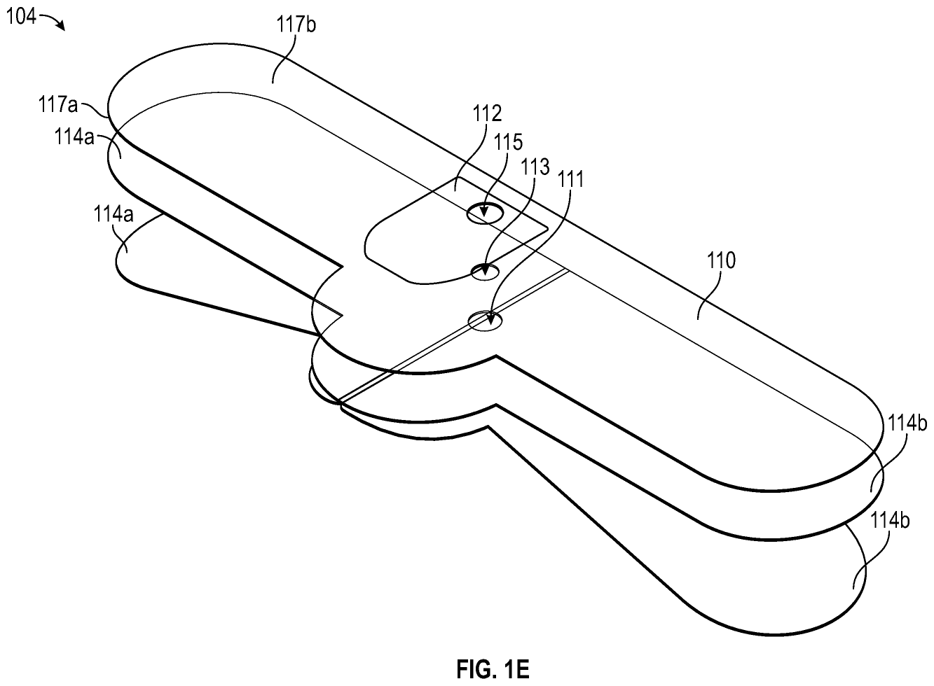

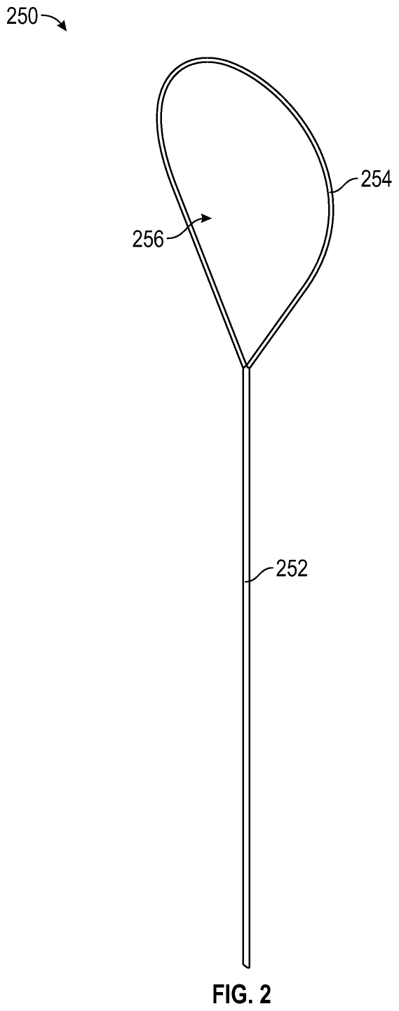

In the illustrated embodiment, (i) the protection layer 114 includes/defines a first opening 111 (e.g., a through-hole or aperture formed between the first removable protection layer 114a and the second removable protection layer 114b), (ii) the contact layer 110 includes a second opening 113, and (iii) the adhesive portion 112 includes a third opening 115 (collectively “openings 111-115”). Referring to FIGS. 1A, 1B, and lE together, the openings 111-115 are at least partially aligned to define a through-hole 118 extending entirely through the dressing assembly 104 between a first surface 117a (e.g., a lower surface) and a second surface 117b (e.g., an upper surface) thereof. The tensioning assembly 102 is configured to be secured to the adhesive portion 114 of the dressing assembly 104 over the through-hole 118. As described in greater detail below with reference to FIGS. 3-4B, one or more sutures can be threaded (e.g., routed, inserted) through the through-hole 118 to the tensioning assembly 102.

In operation of the suture tensioning device 100, the dressing assembly 104 can be placed against the skin of a patient with the protection layer 114 in place. After positioning the openings 111-115 over/at a desired location, such as an incision in the patient, an operator can peel back and remove the first removable protection layer 114a and the second removable protection layer 114a and then adhere the contact layer 110 to the patient.

Referring to FIGS. 1A-1D together, the tensioning assembly 102 can include an actuation member 120 movably (e.g., slidably) coupled to/within a housing 130 having a lower housing portion 132 and an upper housing portion 134. In the illustrated embodiment, the lower housing portion 132 and/or the upper housing portion 134 (e.g., either together or individually) define a channel 131 including a first biasing member mount 136a, a second biasing member mount 136b, a first stop surface 138, and a second stop surface 139. The actuation member 120 can include (i) a press portion 122 extending at least partially outside the housing 130, (ii) an elongate portion 124 extending from the press portion 122 into the housing 130 past the second stop surface 139, and (iii) a flange or mount portion 126 extending from the elongate portion 124 and at least partially through the channel 131. A first biasing member 140a can be operably coupled between the first biasing member mount 136a and the mount portion 126 of the actuation member 120. Likewise, a second biasing member 140b can be operably coupled between the second biasing member mount 136b and the mount portion 126 of the actuation member 120.

The biasing members 140 are configured (e.g., shaped, sized, and/or positioned) to drive the actuation member 120 outward away from the first stop surface 138 in a direction indicated by arrow A in FIG. 1A (e.g., in a horizontal or lateral direction). In some embodiments, the biasing members 140 are compression springs that extend through the channel 131 and that are directly attached to the biasing member mounts 136 and the mount portion 126 via an adhesive, fasteners (e.g., screws), welding, or another suitable attachment mechanism. In other embodiments, the tensioning assembly 102 can include more or fewer of the biasing members 140 (e.g., one, three, four or more) operably coupling the actuation member 120 to the housing 130. In some embodiments, the biasing members 140 can have a spring constant of between about 10-25 pounds per inch (e.g., between about 12-22 pounds per inch, between about 16-18 pounds per inch, about 17 pounds per inch, and so on).

In the illustrated embodiment, the lower housing portion 132 includes/defines a first through-hole 133 (FIG. 1D), the elongate portion 124 of the actuation member 120 includes a second through-hole 123 (FIG. 1C), and the upper housing portion 134 includes a third through-hole 137. The first through-hole 133 and the third through-hole 137 are at least partially aligned with one another (e.g., superimposed over one another) along an axis B shown in FIGS. 1C and 1D (e.g., a vertical axis).

The tensioning assembly 102 is in a first position (e.g., a lock position) in FIGS. 1A-1D in which the biasing members 140 bias the actuation member 120 outward in the direction of arrow A such that the mount portion 126 engages/contacts the second stop surface 139 of the lower housing portion 132. In the first position, the second through-hole 123 through the elongate portion 124 of the actuation member 120 is not aligned with the first and third through-holes 133, 137 of the housing 130. More specifically, the second through-hole 123 is positioned axially away from the axis B such that the second through-hole 123 is not superimposed with the first and third through-holes 133, 137. In operation, the actuation member 120 can be actuated to move the tensioning assembly 102 from the first position to a second position (e.g., a release position; shown in FIG. 4B) in which the second through-hole is aligned with the first and third through-holes 133, 137. More specifically, a user (e.g., a physician, nurse) can press the press portion 122 of the actuation member 120 to drive the actuation member 120 inward in the direction of arrow C shown in FIG. 1B—against the biasing force of the biasing members 140—until the mount portion 126 engages the first stop surface 138 of the lower housing portion 132. In the second position, the second through-hole 123 is at least partially aligned with the first and third through-holes 133, 137 along the axis B. In some embodiments, the movement of the actuation member 120 in the direction of arrow C is generally orthogonal to the axis B.

Referring to FIGS. 1A-1E together, in some embodiments the pad 106 can be coupled to/between the lower housing portion 132 and the adhesive portion 114 of the dressing assembly 104 via an adhesive, fasteners, a snap-fit arrangement, and/or the like. In some embodiments, the pad 106 can be flexible and configured to conform to the skin of a patient during use of the suture tensioning device 100. For example, the pad 106 can be formed from a thermoplastic elastomer (TPE), rubber, silicone, and/or another flexible material. In some embodiments, the adhesive portion 114 of the dressing assembly 104, the pad 106, and the lower housing portion 132 of the housing 130 all have the same generally curved-rectangular planform shape. In other embodiments, the adhesive portion 114, the pad 106, and the lower housing portion 132 can have different shapes (e.g., rectangular, circular, rectilinear, polygonal, irregular) and/or different shapes than one another. In some embodiments, the pad 106 can be omitted and the lower housing portion 132 can be directly attached to the adhesive portion 114 of the dressing assembly 104.

In the illustrated embodiment, the pad 106 includes/defines a through-hole 108 that is aligned along the axis B with (i) the with the first and third through-holes 133, 137 of the tensioning assembly 102 and (ii) the through-hole 118 of the dressing assembly 104. Accordingly, when the tensioning assembly 102 is in the second position with the actuation member 120 pressed inward toward the housing 130, each of the through-holes 108, 118, 123, 133, and 137 are at least partially aligned to define a continuous path through the suture tensioning device 100 for receiving one or more sutures.

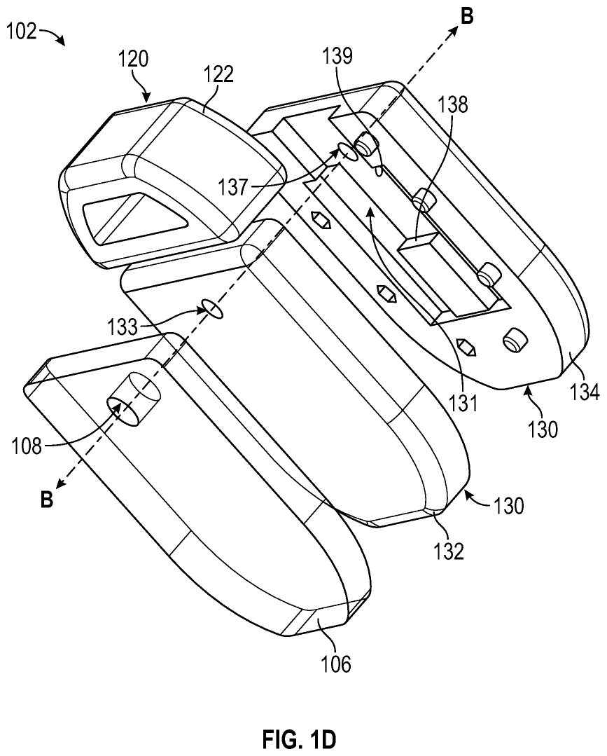

FIG. 2 is an isometric view of a threading tool 250 for use with the suture tensioning device 100 in accordance with embodiments of the present technology. The threading tool 250 can include an elongate portion 252 and a loop portion 254 enclosing/defining an opening 256. In some embodiments, the elongate portion 252 and the loop portion 254 can be formed from a flexible material, such as a nylon monofilament. In some embodiments, the elongate portion 252 can be more rigid than the loop portion 254. For example, the elongate portion 252 can be formed of a different, more rigid material than the loop portion 254 and/or can be made thicker than the loop portion 254. As described in detail below with reference to FIGS. 4A and 4B, the threading tool 250 can be inserted and pulled through the tensioning assembly 102 when the tensioning assembly 102 is in the second position to pull/thread one or more sutures positioned in the opening 256 through the tensioning assembly 102.

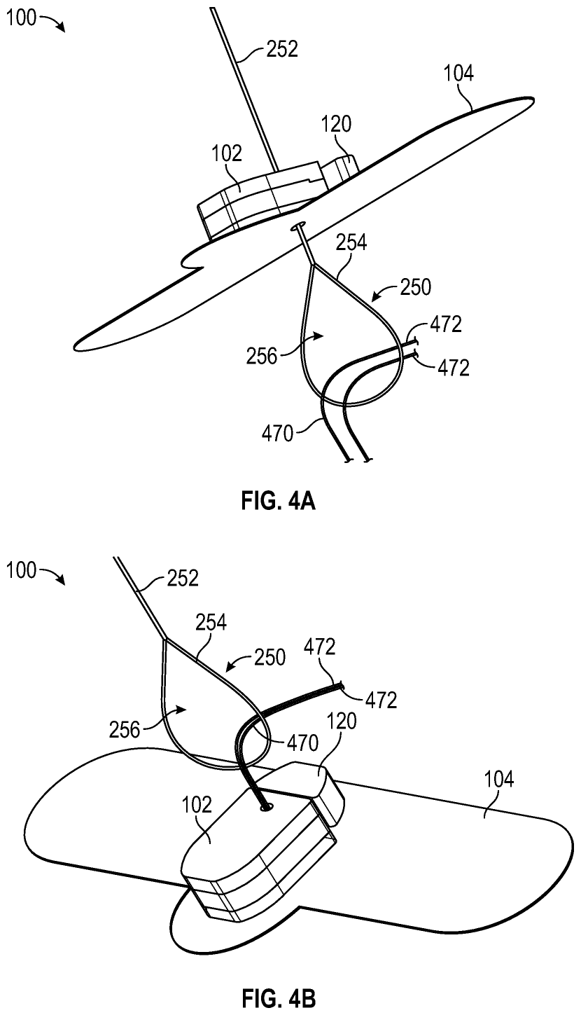

FIG. 3 is a flow diagram of a process or method 360 for operating the suture tensioning device 100 in accordance with embodiments of the present technology. Although some features of the method 360 are described in the context of the embodiments shown in FIGS. 1A-2 for the sake of illustration, one skilled in the art will readily understand that the method 360 can be carried out using other suitable systems and/or devices described herein—for example, using any of the suture tensioning devices described below with reference to FIGS. 6-13B.

At block 361, the method 360 includes threading one or more sutures through the suture tensioning device 100. The sutures can be used to close a puncture in a patient. FIGS. 4A and 4B, for example, are isometric views of the suture tensioning device 100 illustrating threading of a suture 470 therethrough before the dressing assembly 104 has been attached to the skin in accordance with embodiments of the present technology. In some embodiments, the sutures 470 have a U.S.P. size of between about #4-0 to #2. Referring first to FIG. 4A, a user can insert/loop the suture 470 (e.g., end portions 472 of the suture 470) through the opening 256 of the threading tool 250. With the tensioning assembly 102 in the second position, the user can then insert the elongate portion 252 of the threading tool 250 through the through-holes in the dressing assembly 104, the pad 106, and the tensioning assembly 102. For example, the user can depress the actuation member 120 to maintain the tensioning assembly 102 in the second position. Then, referring to FIG. 4B, the user can pull the threading tool 250 entirely through the through-holes in the suture tensioning device 100 to pull the suture 470 therethrough. In other embodiments, the user can thread the suture 470 through the suture tensioning device 100 without using the threading tool 250.

In some embodiments, the suture 470 can be threaded/tied in a figure eight around the puncture. In some embodiments, the suture 470 is not knotted (e.g., in a surgeon's knot) before being threaded through the suture tensioning device 100. The puncture can be an access site into the body of the patient used to carry out a percutaneous medical procedure, such as an intravascular procedure that includes inserting a catheter or medical device into a blood vessel of the patient through the access site. The access site can be a popliteal access site, an internal jugular access site, a femoral access site, or another venous or arterial access site. In some embodiments, the access site can be relatively large, such as those needed to facilitate intravascular procedures utilizing large bore catheters. In some embodiments, the access site can be used to facilitate any of the clot removal procedures described in detail in (i) U.S. Pat. No. 9,700,332, filed Sep. 16, 2016, and titled “INTRAVASCULAR TREATMENT OF VASCULAR OCCLUSION AND ASSOCIATED DEVICES, SYSTEMS, AND METHODS,” (ii) U.S. Pat. No. 10,098,651, filed Apr. 26, 2017, and titled “DEVICES AND METHODS FOR TREATING VASCULAR OCCLUSION,” and/or (iii) U.S. patent application Ser. No. 16/536,185, titled “SYSTEM FOR TREATING EMBOLISM AND ASSOCIATED DEVICES AND METHODS,” filed Aug. 8, 2019, each of which is incorporated herein by reference in its entirety.

At block 362, the method 360 includes coupling the suture tensioning device 100 to the patient. For example, the user can remove the protection layer 116 from the dressing assembly 104 and press the lower layer 110 of the dressing assembly 104 against the skin of the patient around the puncture to adhere the dressing assembly 104 to the patient over/around the puncture. In some embodiments, the dressing assembly 104 can be coupled to the patient before the suture 470 is threaded through the tensioning assembly 102 and/or before the tensioning assembly 102 is coupled to the dressing assembly 104.

At block 363, the method 360 includes adjusting the suture 470 to have a desired/selected tension. For example, the user can continue depressing the actuation member 120 to move the tensioning assembly 102 to the second position, and then pull the suture 470 until a desired tension is achieved. The tension can be sufficient to close the puncture in the patient and maintain hemostasis at the puncture.

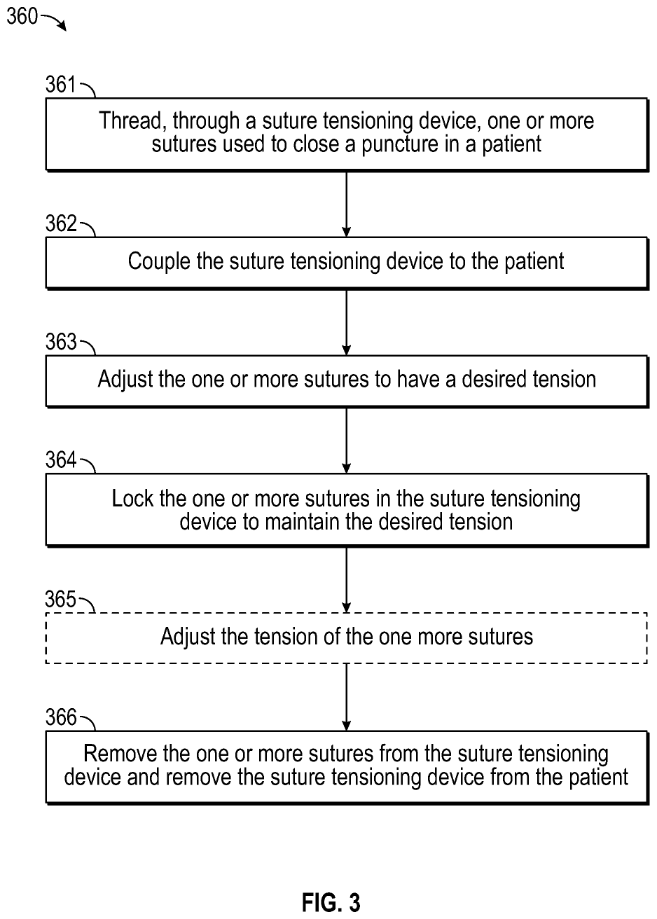

At block 364, the method 360 includes locking the suture 470 in the suture tensioning device 100 to maintain the desired tension in the suture 470. For example, after pulling the suture 470 to have the desired tension, the user can simply release the actuation member 120 to allow the actuation member 120 to return to the first position from the second position, thereby preventing the suture 470 from moving within the tensioning assembly 102. More specifically, FIG. 5 is an enlarged side cross-sectional view of the suture 470 positioned within the tensioning assembly 102 with the actuation member 120 in the first position in accordance with embodiments of the present technology.

Referring to FIGS. 1A-D and 5 together, releasing the actuation member 120 allows the biasing members 140 to drive the actuation member 120 away from the first stop surface 138 toward and/or into engagement with the second stop surface 139, thereby moving the second through-hole 123 out of alignment with the first and third through-holes 133, 137. As the actuation member 120 moves to the first position, a perimeter or edge 527 of the second through-hole 123 engages the suture 470 and pulls the suture 470 through the channel 131 within the housing 130 in the direction of the arrow A (FIG. 1A). Thus, as shown in FIG. 5, the suture 470 traverses through the tensioning assembly 102 along a serpentine path, and the friction/engagement between the suture 470 and the actuation member 120 and the housing 130 can lock/clamp the suture 470 in position—inhibiting or even preventing movement of the suture 470 through the housing 130. That is, the tensioning assembly 102 cinches the suture 470 in the first position to inhibit movement of the suture 470 therethrough.

At block 365, the method 360 optionally includes adjusting the tension of the suture 470. To adjust the tension, the user can depress the actuation member 120 to move the actuation member 120 against the biasing force of the biasing members 140 to the second position, and then advance or retract the suture 470 through the tensioning assembly 102 to decrease or increase the tension, respectively. For example, the user may wish to increase the tension of the suture 470 to increase the constriction force on the puncture to facilitate hemostasis. Conversely, the user may wish to decrease the tension of the suture 470 to accommodate the comfort of the patient. Accordingly, in one aspect of the present technology the suture tensioning device 100 permits the tension in the suture 470 to be simply adjusted by depressing/releasing the actuation member 120 and without disturbing the dressing assembly 104. In contrast, conventional techniques for closing punctures include knotting a suture used to close the puncture to maintain tension in the suture. However, knots prevent the tension in the suture from being easily adjusted—as the knot must be undone and then retied to adjust the tension.

At block 366, the method 360 includes removing the suture 470 from the suture tensioning device 100 and removing the suture tensioning device 100 from the patient. For example, the user can depress the actuation member 120 to move the actuation member 120 to the second position, and then pull the tensioning assembly 102 and/or the dressing assembly 104 away from the patient to remove the suture 470 therefrom. In some embodiments, the tensioning assembly 102 can be decoupled from the dressing assembly 104 and removed from over the suture 470 while the dressing assembly 104 remains on the patient.

FIGS. 6-13B illustrate additional embodiments of suture tensioning devices and tensioning assemblies in accordance with the present technology. The suture tensioning devices described with reference to FIGS. 6-13B can include several features generally similar or identical to the features of one another and/or the features of the suture tensioning device 100 described in detail with reference to FIGS. 1A-5, and can be operated in and function in a generally similar or identical manner. Accordingly, the description of FIGS. 6-13B emphasizes the unique features of the various embodiments. However, one skilled in the art will appreciate that the various embodiments can be combined and/or modified. For example, each of the tensioning assemblies described with reference to FIGS. 6-13B can be coupled to a dressing assembly (e.g., the dressing assembly 104) and/or a flexible pad (e.g., the pad 106), even if not expressly shown.

FIG. 6 is an exploded isometric view of a tensioning assembly 602 in accordance with additional embodiments of the present technology. In the illustrated embodiment, the tensioning assembly 602 includes an actuation member 620 operably coupled to a housing or base 630 via a first biasing member 640a and a second biasing member 640b. More specifically, the first biasing member 640a can be coupled in/between a first biasing member mount 629a on the actuation member 620 and a first channel 636a formed in the base 630. Similarly, the second biasing member 640b can be coupled in/between a second biasing member mount 629b on the actuation member 620 and a second channel 636b formed in the base 630. The base 630 can include a channel 631 configured to slidably receive an elongate portion 624 of the actuation member 620. The base 630 can further include a first through-hole 633 and a second through-hole 637 extending at least partially therethrough and providing access to the channel 631. The first and second through-holes 633, 637 can be at least partially aligned with one another (e.g., along an axis extending therethrough). The elongate portion 624 of the actuation member 620 can include a third through-hole 623.

The biasing members 640 are configured to bias the actuation member 620 away from the base 630 to a first position in which the third through-hole is not aligned with (e.g., is offset from) the first and second through-holes 633, 637. A user can depress the actuation member 620 toward the base 630 against the biasing force of the biasing members 640 to drive the elongate portion 624 of the actuation member 620 through the channel 631 such that the third through-hole 623 is aligned with the first and second through-holes 633, 637. Accordingly, in operation the user can insert one or more sutures through the through-holes 623, 633, 637 when the tensioning assembly 602 is in the second position, and can then release the actuation member 620 to allow the tensioning assembly 602 to return to the first position. In the first position, the actuation member 620 and the base 630 can cinch the sutures (e.g., in the manner described with respect to FIG. 5) to inhibit movement of the sutures through the tensioning assembly 602.

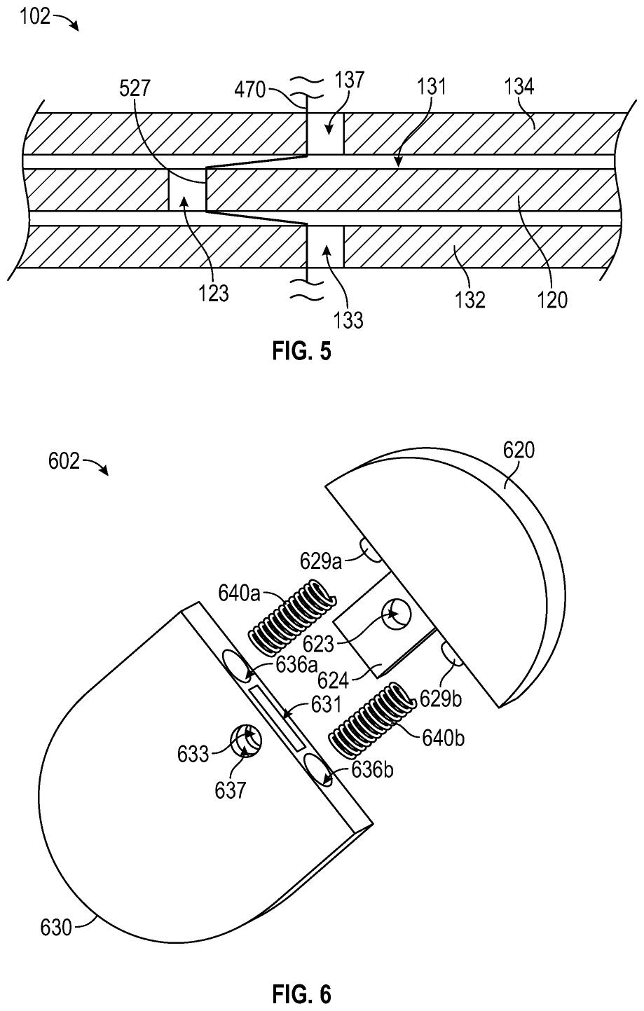

FIGS. 7A and 7B are an exploded isometric view and an isometric view, respectively, of a suture tensioning device 700 in accordance with additional embodiments of the present technology. Referring to FIGS. 7A and 7B together, the suture tensioning device 700 can include a tensioning assembly 702, a dressing assembly 704, and a pad 706 (e.g., a flexible pad). The pad 706 defines a through-hole 708 and is configured (e.g., sized and shaped) to be positioned in an aperture 719 in the dressing assembly 704. For example, the pad 706 can be directly coupled to the skin of a patient through the aperture 719.

In the illustrated embodiment, the tensioning assembly 702 includes a base portion 780 including/defining a channel 781 and configured to be positioned in the through-hole 708 of the pad 706. In other embodiments, the base portion 780 can be coupled to the pad 706 over the through-hole 708. The tensioning assembly 702 further includes a body portion 782 coupled to the base portion 780. The body portion 782 defines a channel 783 that is accessible via a pair of first through-holes 784 (a lower one of the first through-holes 784 is obscured in FIGS. 7A and 7B). The tensioning assembly 702 can further include an actuation member 786 having an elongate portion 785 rotatably positioned within the channel 783. The elongate portion 785 includes a second through-hole 787. The channel 781 of the base portion 780 and the first through-holes 784 of the body portion 782 are at least partially aligned along an axis extending therethrough. The actuation member 786 is rotatable to rotate the second through-hole 787 of the elongate portion 785 into and out of alignment with the first through-holes 784 and the channel 781.

In operation, a user can rotate the actuation member 786 such that the second through-hole 787 is aligned with the channel 781 and the first through-holes 784, and then thread one or more sutures through the channel 781, the lower one of the first through-holes 784, the second through-hole 787, and the upper one of the first through-holes 784. Once the one or more sutures are threaded through the tensioning assembly 702, the actuation member 786 can be rotated to cinch the sutures to inhibit movement of the sutures through the tensioning assembly 702. In some embodiments, the sutures can be wound around the elongate portion 785 of the actuation member 786 to cinch the sutures via the friction between the sutures and the outer surface of the elongate portion 785. In some embodiments, the actuation member 786 can be rotated in a first direction (e.g., clockwise) to increase the tension in the sutures, and in a second direction (e.g., counterclockwise) to decrease the tension in the sutures. Accordingly, in one aspect of the technology the tensioning assembly 702 is actuatable to increase/decrease the tension in the sutures without requiring a user to manually pull/release the sutures through the tensioning assembly 702.

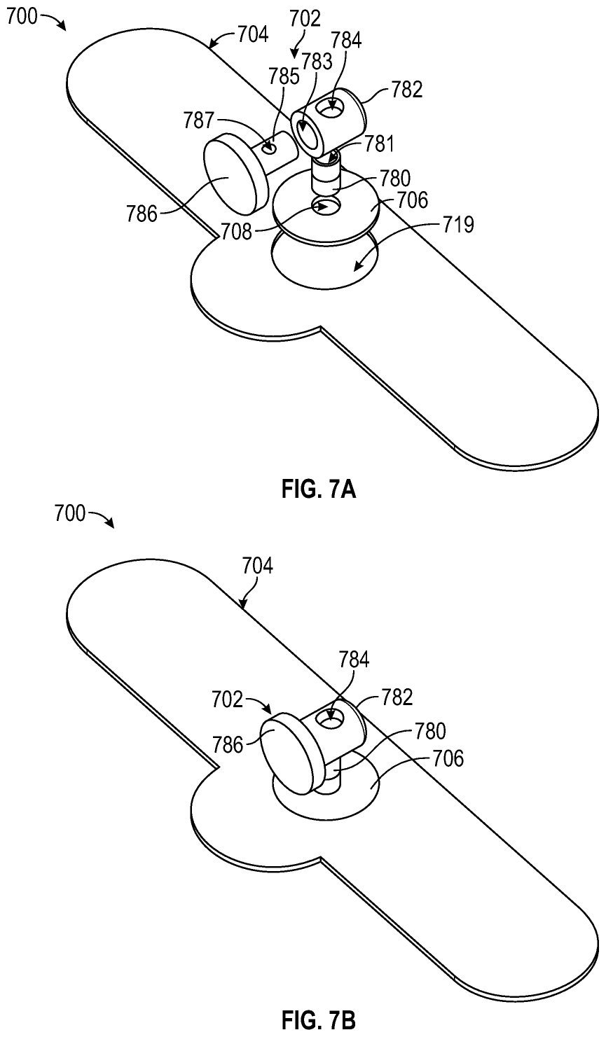

FIG. 8 is an exploded isometric view of a tensioning assembly 802 in accordance with additional embodiments of the present technology. In the illustrated embodiment, the tensioning assembly 802 includes an actuation member 890 (e.g., a lever arm) rotatably coupled to a housing 892. More specifically, the housing 892 can include a base 893 and a sidewall 894 projecting from the base 893, and the actuation member 890 can be an elongate member extending through and supported by the sidewall 894 (e.g., extending through a pair of openings therein). The base 893 can include/define a first through-hole 895, and the actuation member 890 can include a second through-hole 896. In some embodiments, the tensioning assembly 802 can further include a cap 898 having a third through-hole 899 and configured to be coupled to the housing 892 opposite the base 893. When the cap 898 is coupled to the housing 892, the first and third through-holes 895, 899 can be at least partially aligned with one another. The actuation member 890 is rotatable to rotate the second through-hole 896 into and out of alignment with the first and third through-holes 895, 899.

In operation, a user can rotate the actuation member 890 such that the second through-hole 896 is aligned with the first and third through-holes 895, 899, and then thread one or more sutures through each of the through-holes. Once the one or more sutures are threaded through the tensioning assembly 802, the actuation member 890 can be rotated to wind the sutures around the actuation member 890 to cinch the sutures via the friction between the sutures and the outer surface of the actuation member 890. In some embodiments, the actuation member 890 can be rotated in a first direction (e.g., clockwise) to increase the tension in the sutures, and in a second direction (e.g., counterclockwise) to decrease the tension in the sutures. Accordingly, in one aspect of the technology the tensioning assembly 802 is actuatable to increase/decrease the tension in the sutures without requiring a user to manually pull/release the sutures through the tensioning assembly 802.

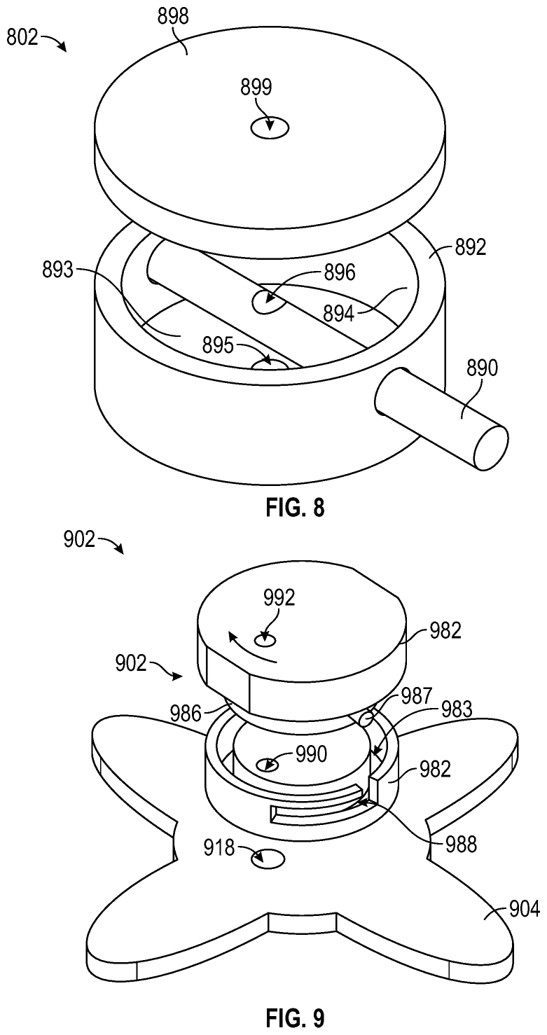

FIG. 9 is an exploded isometric view of a suture tensioning device 900 in accordance with additional embodiments of the present technology. In the illustrated embodiment, the suture tensioning device 900 includes a tensioning assembly 902 configured to be coupled to a dressing assembly 904. The dressing assembly 904 defines a through-hole 918 and can be applied to a patient over and/or proximate to a puncture in the skin of the patient. The tensioning assembly 902 includes a base portion 980 and a cap or lid portion 982 configured to releasably mate with and engage the base portion 980. More specifically, the base portion 980 can include/define an annular groove 983 configured (e.g., shaped, sized, and/or positioned) to receive a flange portion 986 of the lid portion 982. The lid portion 982 can be rotated by a user to rotate the flange portion 986 through the groove 983. In some embodiments, the lid portion 982 further includes a projection 987 extending radially outward from the flange portion 986 and configured to be positioned in a track 988 formed in the base portion 980 (e.g., in a sidewall of the base portion 980). Rotation of the lid portion 982 can bring the projection 987 into contact with end portions of the track 988, thereby limiting the rotation of the lid portion 982. That is, the track 988 can extend only partially around the base portion 980 such that the lid portion 982 is rotatable through an angle less than 360 degrees. In the illustrated embodiment, the base portion 980 includes a first through-hole 990 and the lid portion 982 includes a second through-hole 992 that can be rotated into and out of alignment with the first through-hole 990.

In operation, a user can rotate the lid portion 982 such that the second through-hole 992 is aligned with the first through-hole 990, and then thread one or more sutures through the through-hole 918 in the dressing assembly 904 and the first and second through-holes 992, 994. Once the one or more sutures are threaded through the tensioning assembly 902, the lid portion 982 can be rotated to cinch the sutures in the groove 983 between the flange portion 986 and the base portion 980 to inhibit movement of the sutures through the tensioning assembly 902. In some embodiments, the lid portion 982 can be rotated in a first direction (e.g., clockwise) to increase the tension in the sutures, and in a second direction (e.g., counterclockwise) to decrease the tension in the sutures. Accordingly, in one aspect of the technology the tensioning assembly 902 is actuatable to increase/decrease the tension in the sutures without requiring a user to manually pull/release the sutures through the tensioning assembly 902.

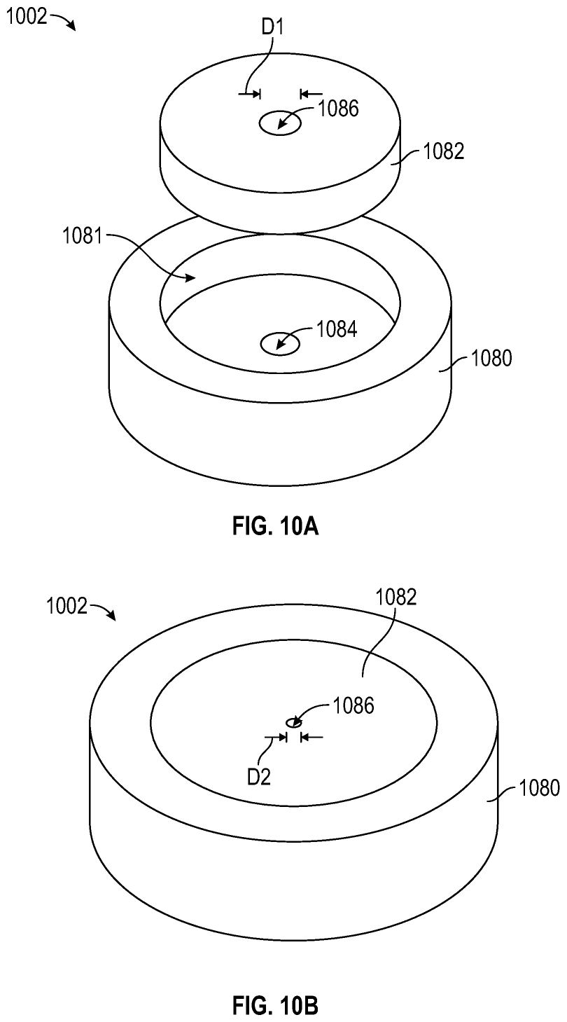

FIGS. 10A and 10B are isometric views of a tensioning assembly 1002 in a first position and a second position, respectively, in accordance with additional embodiments of the present technology. Referring to FIGS. 10A and 10B together, the tensioning assembly 1002 includes (i) a base 1080 defining a recess 1081 and (ii) a compressible member 1082 configured (e.g., shaped and sized) to be inserted into the recess 1081. In some embodiments, the compressible member 1082 can be formed from a material having a softer durometer than the base 1080. Accordingly, the compressible member 1082 can be pushed into the recess 1081 and snugly secured therein via a friction-fit or snap-fit arrangement. In the illustrated embodiment, the base 1080 defines a first through-hole 1084 and the compressible member 1082 defines a second through-hole 1086. In the first position, when the compressible member 1082 is positioned outside of the recess 1081, the second through-hole 1086 can have a first diameter D₁. In the second position, when the compressible member 1082 is compressed inside of the recess 1081, the second through-hole 1086 can have a second diameter D₂ that is smaller than the first diameter D₁. Moreover, the first and second through-holes 1084, 1086 can be at least generally aligned over one another in the second position.

In operation, a user can thread one or more sutures through the first and second through-holes 1084, 1086 when the tensioning assembly 1002 is in the first position. The user can then manually adjust the tension in the sutures before positioning (e.g., snapping) the compressible member 1082 into the recess 1081. The diameter D₂ of the second through-hole 1086 can be small enough that the compressible member 1082 cinches/grips the sutures in the second position to inhibit movement of the sutures through the tensioning assembly 1002. To adjust the tension in the sutures, the user can remove the compressible member 1082 from the recess 1081 and manually adjust the tension.

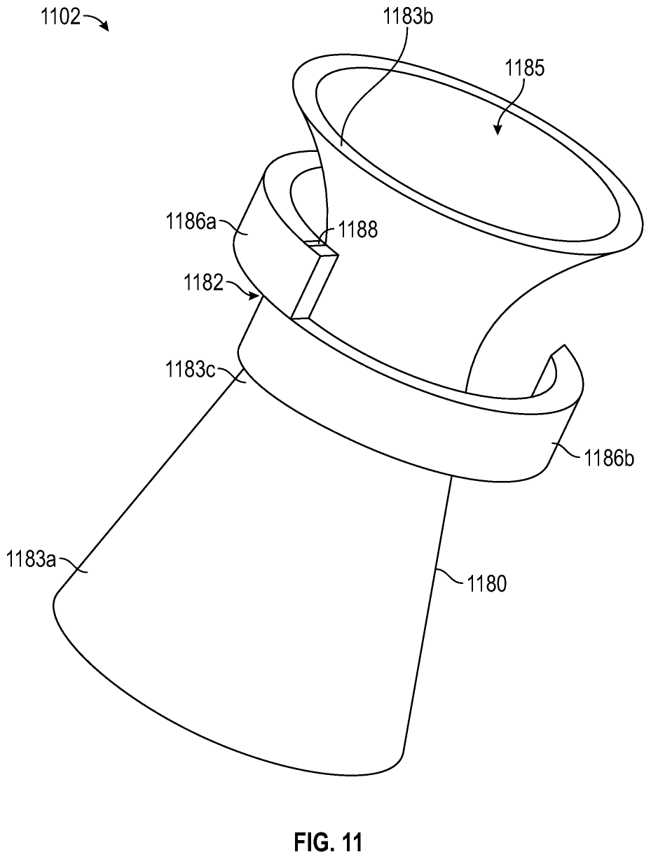

FIG. 11 is an isometric view of a tensioning assembly 1102 in accordance with additional embodiments of the present technology. In the illustrated embodiment, the tensioning assembly 1102 includes a flexible tube 1180 and a clamp 1182 positioned around the flexible tube 1180. The flexible tube 1180 includes a first end portion 1183a, a second end portion 1183b, and a middle portion 1183c extending therebetween. The flexible tube 1180 can define a lumen 1185 extending between the first and second end portions 1183a, b. In some embodiments, a diameter of the lumen 1185 can vary. For example, in the illustrated embodiment the flexible tube 1180 is flared at the first and second end portions 1183a, b such that the diameter of the lumen 1185 is greater at and near the first and second end portions 1183a, b than at the middle portion 1183c. The clamp 1184 can include a first arm 1186a and a second arm 1186b that are pivotally coupled together via, for example, a pin 1188. The clamp 1182 is in a first position in FIG. 11 in which the arms 1186 are pivoted apart from each other. The arms 1186 can each include one or more mating/engagement features (not shown) such that the arms 1186 can be pivoted toward each other and coupled together in a second position (e.g., via a snap-fit arrangement). In the second position, the arms 1186 compress the flexible tube 1180. In the illustrated embodiment, the clamp 1182 is positioned around the middle portion 1183c of the flexible tube 1180. In some embodiments, the diameter of the flexible tube 1180 at the first and second end portions 1183a, b can be greater than a diameter of the clamp 1182 in the first position such that the clamp 1182 does not easily slide off the flexible tube 1180.

In operation, a user can thread one or more sutures through the lumen 1185 when the clamp 1182 is in the first position. The user can then manually adjust the tension in the sutures before moving the arms 1186 of the clamp 1182 to the second position to compress the flexible tube 1180 against/onto the sutures to cinch/grip the sutures to inhibit movement of the sutures through the tensioning assembly 1102. To adjust the tension in the sutures, the user can move the clamp 1182 to the first position illustrated in FIG. 11 and manually adjust the tension.

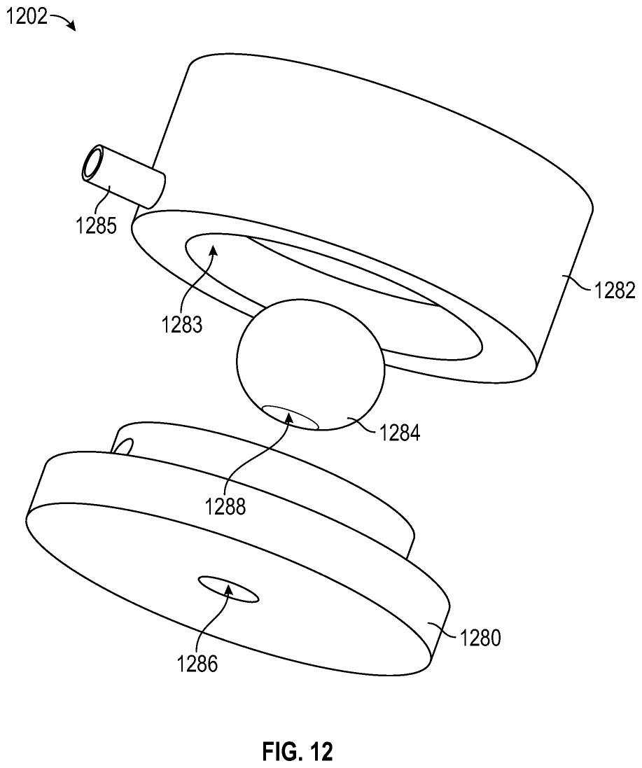

FIG. 12 is an exploded isometric view of a tensioning assembly 1202 in accordance with additional embodiments of the present technology. In the illustrated embodiment, the tensioning assembly 1202 includes a lower portion 1280, an upper portion 1282 coupled to the lower portion 1280 and defining a lumen 1283, and a flexible membrane 1284 positioned in the lumen 1283. The lower portion 1280 can include a first through-hole 1286, the flexible membrane 1284 can include a second through-hole 1288, and the upper portion 1282 can include a third through-hole (obscured in FIG. 12). Each of the through-holes can be at least partially aligned with another. The upper portion 1282 can further include a port 1285 configured to operably/fluidly couple the flexible membrane 1284 with an external pressure source (e.g., a vacuum source such as a syringe). Applying negative pressure to the port 1285 can at least partially collapse the flexible membrane 1284.

Accordingly, in operation, a user can thread one or more sutures through the first through-hole 1286 in the lower portion 1280, the second through-hole 1288 in the flexible membrane 1284, and the third through-hole in the upper portion 1282 without applying negative pressure to the port 1285. The user can then manually adjust the tension in the sutures before applying negative pressure to the port 1285 to collapse the flexible membrane 1284 around the sutures to cinch/grip the sutures to inhibit movement of the sutures through the tensioning assembly 1202. To adjust the tension in the sutures, the user can remove the negative pressure source and manually adjust the tension in the sutures.

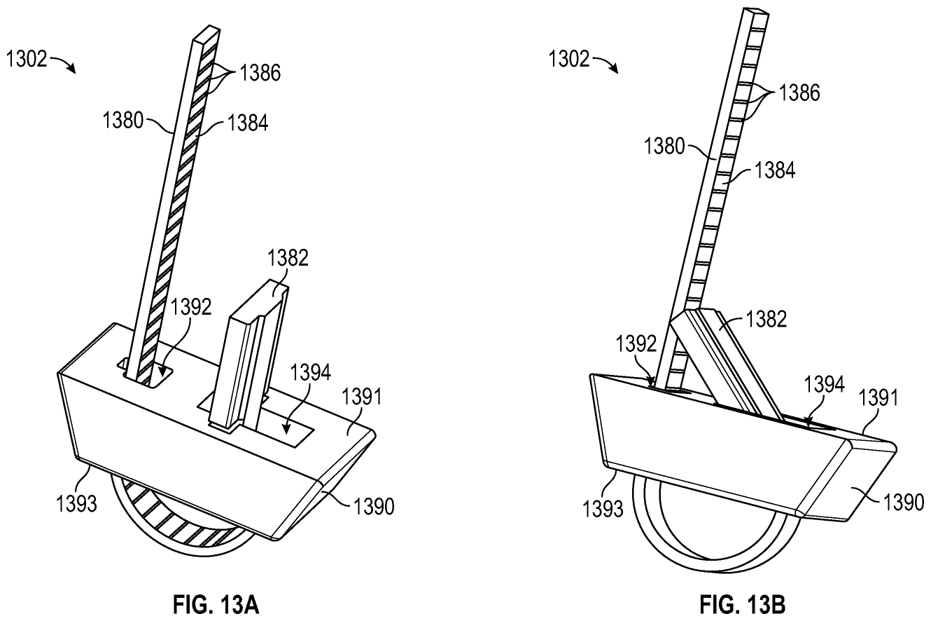

FIGS. 13A and 13B are isometric views of a tensioning assembly 1302 in a first position and a second position, respectively, in accordance with additional embodiments of the present technology. Referring to FIGS. 13A and 13B together, the tensioning assembly 1302 includes an elongate flexible strap 1380 and a base 1390. In the illustrated embodiment, the base 1390 includes a first opening 1392 and a second opening 1394 extending therethrough between a first surface 1391 and a second surface 1393. The strap 1380 can include a tab portion 1382 and a ratchet surface 1384 including a plurality of ridges or ratchet portions 1386. In the illustrated embodiment, the strap 1380 is threaded through the first and second openings 1392, 1394 such that the ratchet surface 1384 faces radially-inward and the tab portion 1382 is positioned near and/or in the second opening 1394 and is accessible at the first surface 1391. In the first positioned shown in FIG. 13A, the tab portion 1382 is separated from (e.g., disengaged from, decoupled from) the ratchet surface 1384. In the second position shown in FIG. 13B, the tab portion 1382 engages the ratchet surface 1384.

In operation, with the tensioning assembly 1302 in the first position, a user can thread one or more sutures along the ratchet surface 1384 of the strap 1380 and through the first opening 1392 (e.g., in a direction from the first surface 1391 toward the second surface 1393). The user can then move the tensioning assembly 1302 to the second position by pressing the tab portion 1382 of the strap 1380 against the ratchet surface 1384 and the suture to secure the suture to the strap 1380 (e.g., against one or more of the ratchet portions 1386). In some embodiments, the user can then adjust the tensioning in the strap by pulling/pushing the strap 1380 through the base 1390, like a zip-tie, until a desired suture tension is reached. To remove the suture, the tensioning assembly 1302 can be returned to the first position and the suture removed from the first opening 1392.

In some embodiments, the tensioning assemblies described in detail herein can have other features and/or the features of the various embodiments can be combined. For example, any of the tensioning assemblies including a rotatable actuation member (e.g., the tensioning assembly 702, the tensioning assembly 802, the tensioning assembly 902, and so on) can utilize a slot rather than a hole for securing the suture to the actuation member. Specifically, for example, the elongate portion 785 of the actuation member 786 of the tensioning assembly 702 described in detail with respect to FIGS. 7A and 7B can include a slot extending at least partially therethrough instead of the second through-hole 787. The slot can receive and secure the suture without requiring that the slot be fully aligned with the channel 781 of the base portion 780 or the first through-hole 784. Similarly, the second through-hole 896 of the actuation member 890 of the tensioning assembly 802 described in detail with respect to FIG. 8 can be replaced with a slot, and so on. In some embodiments, the tensioning assemblies described in detail herein can have one or more locking features for inhibiting movement of the actuation members to, for example, maintain tension in a suture secured thereto/therein.

Several aspects of the present technology are set forth in the following examples:
1. A device for tensioning a suture, comprising:

a housing having a first through-hole; and

an actuator coupled to the housing and having a second through-hole, wherein the actuator is movable relative to the housing from a first position to a second position, wherein the first through-hole and the second through-hole are configured to receive a suture therethrough, and wherein—
when the first through-hole and the second through-hole receive the suture and the actuator is in the first position, the actuator and the housing cooperate to inhibit the suture from moving relative to the housing; and
when the first through-hole and the second through-hole receive the suture and the actuator is in the second position, the actuator and the housing cooperate to permit the suture to move relative to the housing.
2. The device of example 1, further comprising a flexible pad coupled to the housing.
3. The device of example 2 wherein the flexible pad is configured to be placed against skin of a patient and to conform to the skin of the patient.
4. The device of any one of examples 1-3 further comprising a biasing member operably coupled between the actuator and the housing, wherein the biasing member is configured to bias the actuator to the first position.
5. The device of any one of examples 1-4 wherein the first through-hole and the second through-hole are axially offset in the first position, and wherein the first through-hole and the second through-hole are axially aligned in the second position.
6. The device of example 5 wherein the housing and actuator engage the suture in the first position to clamp the suture relative to the housing.
7. The device of any one of examples 1-6 wherein the first through-hole and the second through-hole are aligned along an axis in the second position, and wherein the actuator is movable from the first position to the second position in a direction generally orthogonal to the axis.
8. A suture tensioning device, comprising:

a housing having a first through-hole and a second through-hole, wherein the first and second through-holes are at least partially aligned along an axis;

an actuator slidably coupled to the housing, wherein the actuator includes a third through-hole; and

a biasing member operably coupled between the actuator and the housing, wherein the biasing member is configured to bias the actuator toward a first position in which the third through-hole is offset from the axis, and wherein the actuator is movable to a second position in which the third through-hole is at least partially aligned with the first and second through-holes along the axis.
9. The suture tensioning device of example 8 wherein the first, second, and third through-holes are configured to receive a suture therethrough, and wherein the actuator and the housing engage the suture when the actuator is in the first position to inhibit the suture from moving through the first, second, and third through-holes.
10. The suture tensioning device of example 8 or example 9 wherein the first, second, and third through-holes are configured to receive a suture therethrough, and wherein the actuator and the housing engage the suture when the actuator is in the first position to maintain a tension of the suture.
11. The suture tensioning device of any one of examples 8-10 wherein the biasing member is a first biasing member, wherein the suture tensioning device further comprises a second biasing member operably coupled between the actuator and the housing, and wherein the first and second biasing members are configured to bias the actuator to the first position.
12. The suture tensioning device of any one of examples 8-11, further comprising a flexible pad coupled to a lower portion of the housing, wherein the flexible pad includes a fourth through-hole aligned with the first and second through-holes along the axis, and wherein the flexible pad is configured to be placed against skin of a patient and to conform to the skin of the patient.
13. The suture tensioning device of any one of examples 8-12, further comprising a dressing assembly configured to be attached to skin of a patient around a puncture in the skin, wherein the housing is configured to be releasably coupled to the dressing assembly.
14. The suture tensioning device of any one of examples 8-14 wherein the actuator is movable from the first position to the second position in a direction generally orthogonal to the axis.
15. A method of tensioning a suture used to close a puncture in skin of a patient, the method comprising:

threading the suture through a suture tensioning device;

adjusting a tension of the suture to a desired tension; and

actuating the suture tensioning device to lock the suture in the suture tensioning device to maintain the desired tension.
16. The method of example 15 wherein actuating the suture tensioning device to lock the suture includes releasing an actuator of the suture tensioning device.
17. The method of example 16 wherein adjusting the tension of the suture includes pulling the suture while depressing the actuator.
18. The method of example 16 or example 17 wherein the method further comprises, after releasing the actuator to lock the suture, pulling the suture while depressing the actuator to adjust the tension in the suture from the desired tension to another desired tension.
19. The method of any one of examples 15-18 wherein the method further comprises adjusting the tension in the suture from the desired tension to another desired tension, wherein adjusting the tension in the suture to the other desired tension includes actuating the suture tensioning device to unlock the suture from the suture tensioning device.
20. The method of any one of examples 15-19 wherein the method further comprises positioning a flexible pad of the suture tensioning device against the skin of the patient.

The above detailed descriptions of embodiments of the technology are not intended to be exhaustive or to limit the technology to the precise form disclosed above. Although specific embodiments of, and examples for, the technology are described above for illustrative purposes, various equivalent modifications are possible within the scope of the technology as those skilled in the relevant art will recognize. For example, although steps are presented in a given order, alternative embodiments may perform steps in a different order. The various embodiments described herein may also be combined to provide further embodiments.

From the foregoing, it will be appreciated that specific embodiments of the technology have been described herein for purposes of illustration, but well-known structures and functions have not been shown or described in detail to avoid unnecessarily obscuring the description of the embodiments of the technology. Where the context permits, singular or plural terms may also include the plural or singular term, respectively.

Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Additionally, the term “comprising” is used throughout to mean including at least the recited feature(s) such that any greater number of the same feature and/or additional types of other features are not precluded. It will also be appreciated that specific embodiments have been described herein for purposes of illustration, but that various modifications may be made without deviating from the technology. Further, while advantages associated with some embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

I/We claim:

1. A device for tensioning a suture, comprising:
a housing having a first through-hole; and
an actuator coupled to the housing and having a second through-hole, wherein the actuator is movable relative to the housing from a first position to a second position, wherein the first through-hole and the second through-hole are configured to receive a suture therethrough, and wherein—
when the first through-hole and the second through-hole receive the suture and the actuator is in the first position, the actuator and the housing cooperate to inhibit the suture from moving relative to the housing; and
when the first through-hole and the second through-hole receive the suture and the actuator is in the second position, the actuator and the housing cooperate to permit the suture to move relative to the housing.

2. The device of claim 1, further comprising a flexible pad coupled to the housing.

3. The device of claim 2 wherein the flexible pad is configured to be placed against skin of a patient and to conform to the skin of the patient.

4. The device of claim 1 further comprising a biasing member operably coupled between the actuator and the housing, wherein the biasing member is configured to bias the actuator to the first position.

5. The device of claim 1 wherein the first through-hole and the second through-hole are axially offset in the first position, and wherein the first through-hole and the second through-hole are axially aligned in the second position.

6. The device of claim 5 wherein the housing and actuator engage the suture in the first position to clamp the suture relative to the housing.

7. The device of claim 1 wherein the first through-hole and the second through-hole are aligned along an axis in the second position, and wherein the actuator is movable from the first position to the second position in a direction generally orthogonal to the axis.

8. A suture tensioning device, comprising:
a housing having a first through-hole and a second through-hole, wherein the first and second through-holes are at least partially aligned along an axis;
an actuator slidably coupled to the housing, wherein the actuator includes a third through-hole; and
a biasing member operably coupled between the actuator and the housing, wherein the biasing member is configured to bias the actuator toward a first position in which the third through-hole is offset from the axis, and wherein the actuator is movable to a second position in which the third through-hole is at least partially aligned with the first and second through-holes along the axis.

9. The suture tensioning device of claim 8 wherein the first, second, and third through-holes are configured to receive a suture therethrough, and wherein the actuator and the housing engage the suture when the actuator is in the first position to inhibit the suture from moving through the first, second, and third through-holes.

10. The suture tensioning device of claim 8 wherein the first, second, and third through-holes are configured to receive a suture therethrough, and wherein the actuator and the housing engage the suture when the actuator is in the first position to maintain a tension of the suture.

11. The suture tensioning device of claim 8 wherein the biasing member is a first biasing member, wherein the suture tensioning device further comprises a second biasing member operably coupled between the actuator and the housing, and wherein the first and second biasing members are configured to bias the actuator to the first position.

12. The suture tensioning device of claim 8, further comprising a flexible pad coupled to a lower portion of the housing, wherein the flexible pad includes a fourth through-hole aligned with the first and second through-holes along the axis, and wherein the flexible pad is configured to be placed against skin of a patient and to conform to the skin of the patient.

13. The suture tensioning device of claim 8, further comprising a dressing assembly configured to be attached to skin of a patient around a puncture in the skin, wherein the housing is configured to be releasably coupled to the dressing assembly.

14. The suture tensioning device of claim 8 wherein the actuator is movable from the first position to the second position in a direction generally orthogonal to the axis.

15. A method of tensioning a suture used to close a puncture in skin of a patient, the method comprising:
threading the suture through a suture tensioning device;
adjusting a tension of the suture to a desired tension; and
actuating the suture tensioning device to lock the suture in the suture tensioning device to maintain the desired tension.

16. The method of claim 15 wherein actuating the suture tensioning device to lock the suture includes releasing an actuator of the suture tensioning device.

17. The method of claim 16 wherein adjusting the tension of the suture includes pulling the suture while depressing the actuator.

18. The method of claim 16 wherein the method further comprises, after releasing the actuator to lock the suture, pulling the suture while depressing the actuator to adjust the tension in the suture from the desired tension to another desired tension.

19. The method of claim 15 wherein the method further comprises adjusting the tension in the suture from the desired tension to another desired tension, wherein adjusting the tension in the suture to the other desired tension includes actuating the suture tensioning device to unlock the suture from the suture tensioning device.

20. The method of claim 15 wherein the method further comprises positioning a flexible pad of the suture tensioning device against the skin of the patient.
